(19) United States
(12) Patent Application Publication
(10) Pub. No.: US 2010/0112150 A1
(43) Pub. Date: May 6, 2010

(54) Methods and apparatuses for obtaining infusions

(75) Inventors: Luciano Navarini, Trieste (IT); Riccardo De Luca, Gorizia (IT); Luca Mastropasqua, Trieste (IT); Bruno Dellapietra, Duino Auristina (TS) (IT); Furio Suggi Liverani, Trieste (IT)

Correspondence Address: NIXON & VANDERHYE, PC, 901 NORTH GLEBE ROAD, 11TH FLOOR, ARLINGTON, VA, 22203, US

(73) Assignee: ILLYCAFFE' S.P.A., Trieste (IT)

(21) Appl. No.: 12/654,849

(22) Filed: Jan. 6, 2010

Related U.S. Application Data
(63) Continuation of application No. 11/921,468, filed on Dec. 3, 2007.

(30) Foreign Application Priority Data
Jun. 3, 2005 (IT) MO2005A000140

(51) Int. Cl. G01N 33/14; A47J 31/00

(52) U.S. Cl. 426/231; 99/285

(57) ABSTRACT

An apparatus for obtaining beverages by infusion comprises an indicating device arranged for indicating that a preset infusion time interval has passed; a method for obtaining beverages by infusion comprises imbibing with water at a desired temperature a dose of a substance or of a mixture of substances from which beverages have to be obtained, and indicating the duration of the infusion through a suitable indicating device indicating that a preset infusion time interval has passed; a timing device is provided with a hooking element arranged for hooking said timing device to an apparatus for preparing beverages by infusion.

This application is a continuation of Ser. No. 11/921,468 filed Dec. 3, 2007, which in turn is a continuation of PCT International Application No. PCT/EP2006/062742 filed May 30, 2006. PCT/EP2006/062742 claims priority to IT Application No. MO2005A000140 filed Jun. 3, 2005. The entire contents of this application are incorporated herein by reference.

The invention relates to improved methods and apparatuses for obtaining various kind of infusions.

It is very common to prepare beverages by infusion both for the wide kinds of beverages that can be prepared and for the simplicity of the preparation of such beverages.

With this method tea-, or coffee-based beverages, or infusions of herbs, flowers, fruits, or of phytotherapic mixtures can be prepared, i.e. can be prepared beverages having features even very different from one another.

Beverages obtained by infusion not always have the features required by a user thereof, for instances, coffee and/or tea beverages can be too strong or too little strong, i.e. too much or too little rich in caffeine/theine and/or in other aromatic substances.

Furthermore, could be obtained beverages having not good organoleptic features, and which can be not pleasant for the user.

Too strong or too little strong coffee and/or tea beverages can have undesired effect to the user that recognise the actual features of the prepared beverage only at the moment of the tasting of the beverage, or even at the moment in which the user feels such effects on himself.

This become even more important in the case in which the beverages obtained by infusion are phytotherapic preparations, since incorrectly preparing such a beverage implies that the beverage is devoid of the active principles that the user thought to find therein and to take with the beverage, and implies also that such beverages have no beneficial effect on the organism of the user.

This problem becomes even more important because spreads more and more the use of phytotherapic beverages for treating different diseases, for example diseases of the gastrointestinal, respiratory, cardio-circulatory system, sleep disorders, etc.

Furthermore a user does not usually have the criteria for evaluating the features of the prepared beverage before tasting it, in fact the decision of considering a beverage ready for being tasted is based on considerations due to the appearance of the obtained beverage, and in particular on the colour thereof.

This implies that the real organoleptic features of the beverage and/or the actual presence in the beverage of the desired active principles can not be evaluated, since the colour of the beverage greatly depends on the particular infused substances and can thus lead the user to erroneous considerations.

In addition, certain kinds of apparatuses for preparing beverages by infusion are provided with a non-transparent container arranged for receiving the prepared beverage, therefore also it is not possible to make the appearance's based valuations for considering the beverage ready unless by opening the apparatus, therefore such valuations are made basing on personal, irreproducible, and unreliable considerations made by the user.

An object of the invention is to improve apparatuses and methods for obtaining beverages by infusion.

Another object is to obtain apparatuses and methods for obtaining beverages by infusion, such as coffee and/or tea beverages, infusions of herbs, and/or of flowers, decoctions, allowing beverages having great organoleptic features to be obtained.

Still another object is to provide simple and economic apparatuses allowing a coffee beverage having great organoleptic features to be obtained.

Still another object is to obtain apparatuses and methods for obtaining beverages by infusion allowing beverages with preset properties and desired effects on an organism of a user to be obtained.

Still another object is to obtain apparatuses and methods for obtaining beverages by infusion allowing to a user to choose on his liking, and to change, from time to time according his particular desires, the final features of the obtained beverages, and thus the effects of such beverages on his organism.

In a first aspect of the invention, it is provided an apparatus for obtaining beverages by infusion comprising an indicating device arranged for indicating that a fixed infusion time has passed.

The indicating device can be properly regulated and programmed by a user.

The indicating device can be properly programmed by a user for regulating the infusion time.

In a version, said indicating device comprises a switch arranged for activating/deactivating said device so as to automatically regulate the infusion time.

In another version, said indicating device comprises sensing devices arranged for sensing a fixed parameter and for automatically activating/deactivating said indicating device.

Said sensing devices may comprise temperature sensors sensing the temperature of said apparatus and/or level sensors sensing the level of the liquid in said apparatus, and/or colorimetrical sensors sensing the coloration of the obtained beverage in said apparatus, for example by means of transmittance sensing, and/or conductibility sensors, or still other sensors arranged for sensing desired parameters of the obtained beverage bound to the particular solutes present in the beverage and to their concentration. The sensors are suitable for automatically activating said indicating device in consequence of the actual sensed value of the parameter.

In a further version, said indicating device comprises warning elements arranged for warning a user that the expected infusion time has been reached.

The warning elements can comprise acoustic warning elements emitting a warning sound signal and/or optical warning elements emitting a warning light signal.

In another version, said indicating device comprises a hooking element arranged for movably hooking said indicating device to said apparatus.

In a still another version, said indicating device comprises a first device portion rigidly fixed to the apparatus, and a second device portion that can be movably associated to said first device portion.

In a still another version, it is also provided a frothing device arranged for frothing the obtained beverage.

In a further version, it is also provided a heating device for heating the water in the apparatus.

Preferably, the heating device can be provided with a temperature regulating device for regulating the temperature of the water in the apparatus.

In this way, it is possible to heat the water in the apparatus to the desired temperature, and to pour in the apparatus the powder or the substance, or the mix of substances, from which the beverage has to be obtained, only after the desired temperature has been reached.

It is also possible to choose the temperature of the water according to the specific beverage to be prepared and to the particular desires of the user.

It also possible to maintain the water almost at the same temperature during the entire infusion time.

The desired value of the temperature of the water, and the desired infusion time, can be set by a user according to the specific beverage to be prepared and to the particular desires of the user.

In a second aspect of the invention, it is provided a method for obtaining beverages by infusion comprising imbibing a dose of a substance, or of a mixing of substances, from which said beverages have to be obtained with water at a desired temperature, and indicating the duration of the infusion by means of an indicating device indicating that a fixed infusion time has passed.

In a version said indicating comprises regulating the duration of the infusion.

In a third aspect of the invention, it is provided for a timing device provided with a hooking element arranged for hooking said timing device to an apparatus for preparing beverages by infusion.

In a fourth aspect of the invention, it is provided for the use of a timing device for preparing beverages by infusion.

In a fifth aspect of the invention, it is provided a base comprising a connecting element for connecting said base to an apparatus for preparing beverages by infusion and with a heating device for heating a content of said apparatus.

In a version, said base comprises a programming device for programming the functioning of the apparatus.

In a further version, said programming device comprises a temperature regulating device for regulating the temperature of the water in the apparatus.

In a still further version, said programming device comprises a time regulating device for regulating the infusion time.

In another version, said base comprises an indicating device for indicating that a preset infusion time has passed.

In a still another version, said base comprises a switch arranged for activating/deactivating said base so as to automatically regulate the infusion time and/or the temperature of the water in the apparatus.

In another version, said base comprises a sensing device arranged for sensing a fixed parameter of the water in the apparatus and for automatically activating/deactivating said base.

In a still another version said base comprises a frothing activating device arranged for activating a frothing device provided in the apparatus.

Owing to these aspect of the invention, it is possible to obtain a beverage having organoleptic features, or therapeutic and beneficial effects, and desired properties.

Furthermore, owing to the invention, a user can choose from time to time, following his particular tastes or desires, to obtain beverages having particular desired features and, in case, to reproduce such features in subsequent preparations of beverages.

In fact, by varying the infusion time in hot water of the various substances from which the beverage has to be obtained, the features and the quality of the infusion are changed, the substances being in powder form, or minced, or packed in bags.

A too short infusion time does not enable, in fact, the aromatic substances present in the powder, or in the bag, to be solubilized, or to be solubilized only within certain limits. This implies that a beverage is obtained that is not aromatic, and thus unpleasant for a user, tasteless, and, in the case of phytotherapic preparations, a beverage lacking in the solutes with therapeutic effects is obtained.

On the other hand, a too long infusion time makes the beverage too strong, this can cause undesired effects on a user, and can make a beverage rich of substances, mostly undesired, to be obtained, such substances require long infusion time, can have unwelcome effects on the organism of a user and would not be extracted in an optimal infusion time.

Furthermore, even with long infusion time, a beverage with low organoleptic features and thus unpleasant for a user is obtained.

Furthermore, too long infusion time cause a considerable fall in the temperature of the obtained beverage, this implies a considerable drop of the pleasantness associated to the beverage.

To overcome this last drawback, a user would heat said beverage so getting further worse the organoleptic features thereof.

Really for the importance of the infusion time for obtaining beverage with high organoleptic features, in the packaging of the substances from which such beverages have to be obtained, the optimal infusion time is specified, or such infusion time is indicated and recommended directly by a herbalist or by a person in charge of the preparation and of the sale of herbal and/or phytotherapic compositions for obtaining beverages having the required effects by the user. Nevertheless, usually, it is very difficult for a user, using the known apparatuses to reckon such indications, and the user considers ready the prepared beverage when it has assumed a particular coloration, or when it occurs to the user that he has prepared a beverage and provides for separating the substance from the hot water.

The invention will be better understood and implemented with reference to the attached drawings that show an embodiment thereof by way of non-limitative example in which:

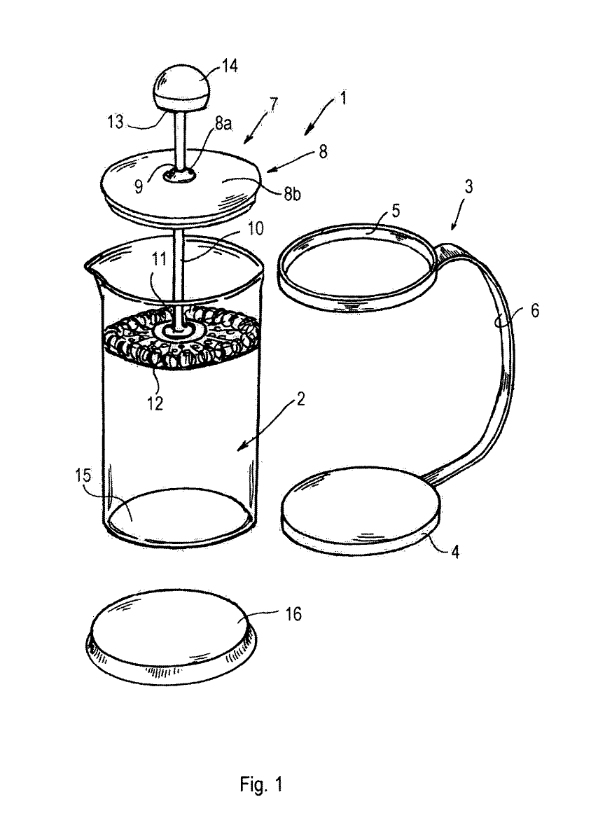

FIG. 1 is an exploded schematic view of a traditional apparatus for obtaining coffee beverages by infusion.

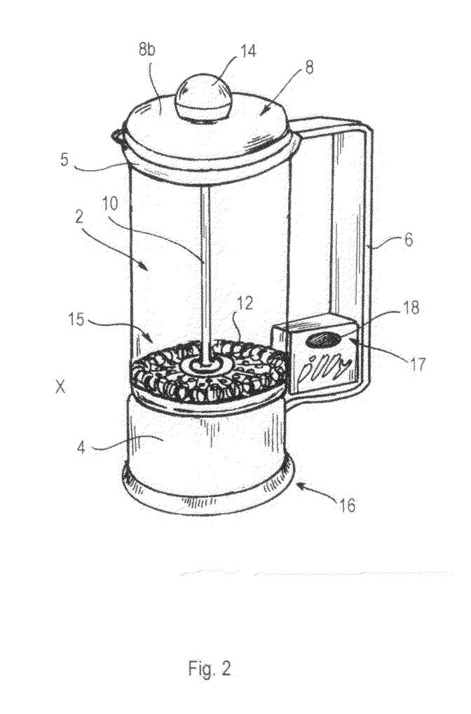

FIG. 2 is a schematic view of a traditional apparatus for obtaining coffee beverages by infusion provided with a device according to the invention;

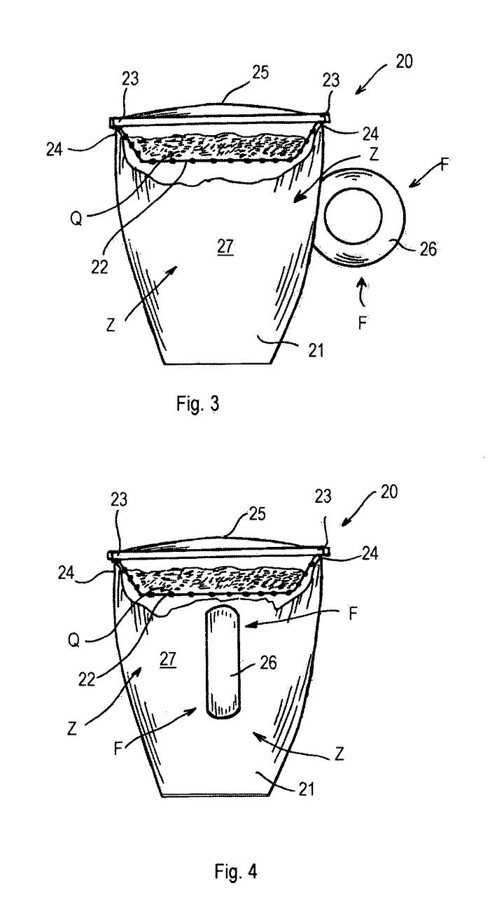

FIG. 3 is a partially sectioned schematic view of an apparatus for infusions provided with a device according to the invention in a possible position thereof;

FIG. 4 is a view like the one in FIG. 3 highlighting another possible different position of the device according to the invention;

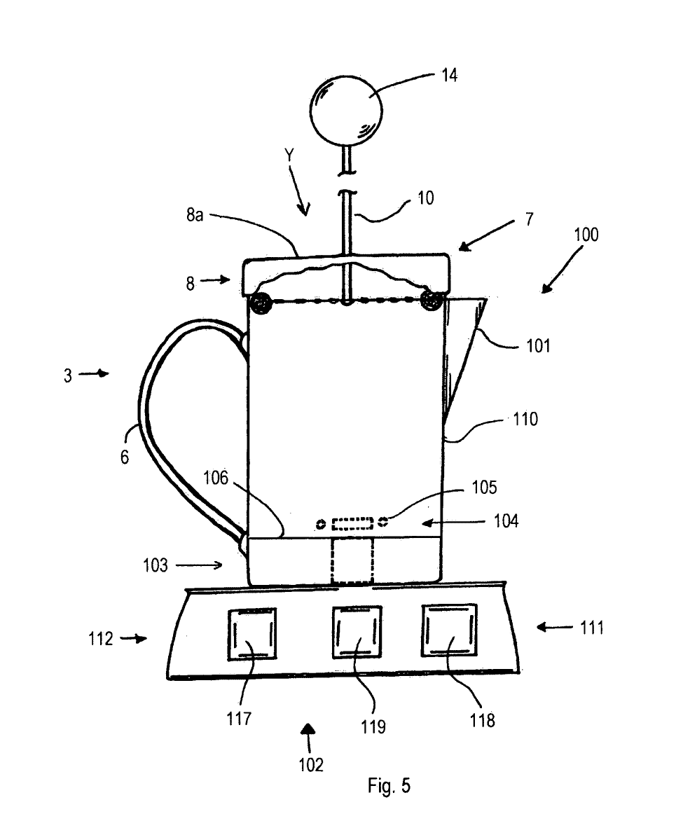

FIG. 5 is a schematic view of an apparatus for obtaining beverages by infusion provided with a base provided with regulating and/or indicating device.

With reference to FIG. 1, 2, 5, it is shown an apparatus 1 for preparing a beverage by infusion, for example a coffee based beverage, comprising a container 2 suitable for containing a preset quantity of beverage to be prepared, the container 2 being preferably made in transparent material, for example glass, pirex, or Plexiglas.

A handgrip 3 can be stably or movably fixed to the container 2, the handgrip 3 being suitable for allowing the apparatus 1 to be calmly handled even when hot water for preparing the beverage has been poured into the container 2. The handgrip 3 preferably comprises a base portion 4 into which the container 2 is accommodated, and that is made in insulating material so as to avoid a surface on which the apparatus 1 is rested to be damaged.

The handgrip 3 comprises an annular portion 5 so shaped that the container 2 can be slidably inserted therein, and a handle portion 6 interposed between the base portion 4 and the annular portion 5 and suitable to be grasped by a user.

The apparatus 1 furthermore comprises a lid 7 provided with a covering portion 8 so shaped as to rest and to close an upper portion of the container 2 and provided in a central zone 8a thereof with a hole 9 inside which a plunger 10 slides. At a first end 11 of the plunger 10, a filter 12 is provided and at a second end 13 of the plunger 10, located at the opposite side in relation to the first end 11, it is provided a knob 14 suitable for making up the handgrip of the lid 7.

The first end 11 of the plunger 10 is located, when the covering portion 8 is resting on the container 2, inside the container 2.

A user by gripping the knob 14 can lower/raise the plunger 10 so as to move the plunger 10 from a rest position Y, shown in FIG. 5, in which the filter 12 is located directly under the covering portion 8, to a filtering position X, shown in FIG. 2, in which the filter 12 is located at the maximum possible distance from the covering portion 8 and the knob 14 is resting on an upper surface 8b of the covering portion 8, and vice versa.

The container 2 can also be provided, as indicated in FIG. 5, with a spout 101 for pouring the prepared beverage in suitable vessels, for example cups, from which the user can taste the beverage.

As it is shown in FIG. 1, the apparatus 1 can be rested on a base 16 made in insulating material and so shaped as to shapingly couple to the base portion 4 of the handgrip 3.

In this way, it is possible, after resting the apparatus 1 on the base 16, to position the base 16 in any desired surface, without risks of damaging said surface.

A user desiring to prepare a beverage using the apparatus 1, will provide at first for positioning a desired quantity of powder of the substance from which the beverage has to be obtained, for example coffee, on a bottom portion 15 of the container 2, therefore, he will pour hot water, nearly boiling, on such powder, and will position the lid 7 in its rest position on the container 2 so as to close the container 2 and to avoid excessive heat losses to the outside.

After a certain time interval, i.e. when the user will consider the beverage ready, he will provide to lower the plunger 10 so as to progressively lower the filter 12 so as to bring it in contact with the coffee powder, and to squeeze the coffee powder thus extracting the aromatic substances therefrom, and separating it from the liquid beverage.

To such apparatus, indicating devices can be fixed, as will be better disclosed in the followings, for indicating the real passed infusion time, and/or regulating devices, for example timers, for regulating the infusion time and, thus, for obtaining beverages with great organoleptic substances. Keeping the plunger in the filtering position X, it would be possible to pour out from the container 2 the obtained beverage into a suitable vessel for tasting it.

The apparatus shown in FIG. 2, is completely analogous to that in FIG. 1, therefore the corresponding parts have been indicated with the same numerical references, and it is different from the apparatus in FIG. 1 for the presence of a timer 17 located on the handle portion 6 of the apparatus 1.

The timer 17 enables to a user to set out the desired infusion time of the coffee powder into the water, basing on his particular desires and tastes.

The timer 17 is provided with a starting switch 18 suitable for operating the timer 17.

In a version not shown, the timer 17 can be provided with temperature sensors sensing the temperature in the container 2 and automatically activating the timer 17 when a desired temperature value has been reached, i.e. when hot water is poured into the container 2.

In another version, not shown, the timer 17 can be provided with level sensors sensing the liquid level into the container 2 and activating the timer 17 when a desired level value in the container 2 has been reached, i.e. when hot water is poured into the container 2 for preparing the beverage.

The timer 17 can be further provided with sound and/or light warning elements, suitable for emitting a warning signal when the infusion time previously set out by the user has been passed.

In other versions not shown, can be further provided colorimetric sensors sensing the coloration of the beverage obtained in the apparatus, for example by means of measurements of the transmittance of the beverage, or can be further provided conductivity sensors that senses the conductivity of the obtained beverage, and thus detect the concentration of the solutes present in the beverage.

Also other sensors can be provided for sensing preset parameters of the beverage depending on the particular solutes present into the beverage and to their concentration. The sensor being able, following the particular measurement made, to automatically activate/deactivate the timer 17, or to emit a particular warning signal for the user, when the beverage has reached desired values of the corresponding parameters measured by the particular sensors.

In case, the timer 17 can be programmed in advance with a preset number of infusion time corresponding to different beverages, both by the user and by the builder.

For example, once a user has decided the particular beverage he wants to prepare, and the particular features he desires to find the prepared beverage, i.e. a strong beverage or a weak beverage, he can program the timer 17, or if the timer 17 had already been programmed, can choose the infusion time corresponding to his desires, as will be better explained in the followings, and prepare a beverage having exactly the desired features.

The timer 17 can be located in any suitable zone of the apparatus 1, can be, for example fixed to the container 2, or to the lid 7, both movably and stably. In the first case, the timer is provided with movable hooking elements for hooking/unhooking the timer to/from the apparatus 1.

The hooking elements can be shaped according to any desired known shape, in relation to the chosen hooking position and to the characteristics of the apparatus 1 and/or of the timer 17. The hooking elements can, for example comprise parts in Velcro, tongs, pliers, elastic bands, magnets, etc.

In case in which, the timer 17 is stably fixed to the apparatus 1, it is made so as to be not damaged during the washing of the apparatus 1.

The timer 17 can be connected to any suitable apparatus for preparing infusions, for example the apparatus shown in FIG. 1, or a teapot, a kettle, into which desired quantities of powder of substances from which the beverage has to be extracted, for example directly at inside of a strainer, pre-dosed bags, or also in a common cup into which pre-dosed doses of substances, as coffee wafers, or bags of tea, or camomile, or herbal infusion, etc., or also in tisane-pot, as that showed in FIGS. 3 and 4.

In this way, it is possible to obtain beverages with high organoleptical features even using known apparatuses.

The afore described apparatus 1 is particularly suitable for obtaining coffee based beverages starting from coffee powder, but the above drafted considerations may be equally applied to apparatuses working with pre-dosed quantities of substances from which beverages have to be obtained, for example single-dose tea and/or camomile bags.

Apparatus 1 can be further provided with a chamber arrangement for conserving the temperature inside the apparatus, so as to keep the temperature of the beverage substantially constant even for a long time, and, thus to allow a user to prolong the tasting of the beverage.

The apparatus 100 shown in FIG. 5, is almost analogous to that in FIG. 2, therefore the corresponding parts have been indicated with the same numerical references.

The apparatus 100 comprises a base 102 shaped as to shapingly coupling with the container 2.

In case, the base 102 can be provided in a surface zone thereof arranged to receive the container 2, with a coupling element, shaped as to shapingly coupling with a respective coupling element provided on the container 2, so to improve the coupling between the base 102 and the container 2.

The base 102 is provided with a heating device 103, schematically indicated in FIG. 5, for example a resistance, arranged for heating the water in the container 2, and with a connecting element for connecting the base 102 to the electric network for activating the resistance.

The resistance can be shaped in any suitable manner so as to obtain an efficient heating of the water in the container 2. The heating device 103 may be preferably provided in a zone of the base 102 arranged to go in use in contact with the container 2, so as to improve the heat transfer from the heating device 103 to the water.

As shown in FIG. 5, the heating device 103 is provided in an upper zone of the base 102 so as to speed up the heating of the water in the container 2.

In a version not shown, the base 102, can be shaped so as to partially envelop a side surface 110 of the container 2, so as to widen the surface of the container 2 through which the heat is transferred from the heating device 103 to the water in the container 2.

The base 102, further comprises a controlling arrangement 111 arranged for controlling and programming the functioning of the apparatus 100.

The controlling arrangement 111 comprises indicating devices 112 arranged for indicating the actual value of desired functioning parameters of the apparatus 100.

The indicating devices 112 comprise a timer 117 arranged for indicating the real infusion time, and/or for regulating the infusion time, a temperature controlling device 118 for controlling the temperature of the water in the container 2, and a frothing controlling device 119 for controlling a magnetic stirrer 104 for frothing the beverage in the container 2 for producing foam.

Using the timer 117, a user can program the desired infusion time depending on the particular beverage to be prepared, and/or the desired features of the beverage, and/or his particular tastes, and then activate the timer 117 so that the remaining infusion time and/or the passed infusion time could be indicated.

The timer 117 can be provided with a warning element arranged for warning a user that a preset interval of time has passed.

The base 102 can be also provided with a central unit, in which a certain number of infusion times can be stored, by the user or by a builder, so that a user desiring to prepare a beverage can choose the relevant stored infusion time and thus activate the timer 117.

The frothing controlling device 119 controls and drives a magnetic stirrer 104 for frothing the beverage in the container 2 for producing foam.

The magnetic stirrer 104 comprises a stirrer 105, located on a bottom part 106 of the container 2 so as to be at least partly immersed into the water, and a driving part provided into the base 102, and thus not shown, arranged for driving the stirrer 105 so as to move it into the beverage for producing foam.

In versions not shown, frothing devices of different type can also be used, for example a whisk driven by an electrical motor.

The container 2 could be devoid of at least a portion of the bottom part and so shaped as to tightly coupling to the base 102.

The frothing device may be provided on an upper part of the base 102, so that when the container 2 is tightly coupled to the base 2, the frothing device is at least partly immersed in the liquid in the container 2.

Thereafter the frothing device can be activated for frothing the liquid in the container 2 so producing foam.

The tight connection between the base 102 and the container allows no leakages of liquid from the container 2 to occur.

The temperature controlling device 118 controls the temperature of the water in the container 2, and it is provided with a temperature indicating element arranged for indicating the actual value of the temperature of the water and/or of the beverage in the container 2.

In this way, it is possible to switch on the apparatus 100 so as to activate the heating device 103 for heating the water in the container 2, and to pour in the container 2 the substance from which the beverage has to be obtained only when the desired infusion temperature has been reached.

The temperature controlling device 118 is connected with temperature sensors arranged for sensing the temperature of the water, so as to activate/deactivate the heating device 103.

Therefore, it is possible to automatically switch off the heating device 103 when the desired infusion temperature has been reached, and it is also possible to act on the heating device 103 so as to activate/deactivate the heating device 103 for keeping almost constant the temperature of the water in the container 2 for the entire infusion time.

In a version, the temperature controlling device 118 acts directly on the heating device 103 without the presence of the temperature sensors, so as to activate/deactivate the heating device basing on preset functioning values.

A user can program the desired infusion temperature depending on the particular beverage to be prepared, and/or the desired features of the beverage, and/or his particular tastes, and then activate the temperature controlling device 118 so that the heating device 103 can correspondingly be activated/deactivated.

If the base 102 is provided with a central unit, a certain number of infusion temperatures can be stored therein, by the user or by a builder, so that a user desiring to prepare a beverage can choose the relevant stored infusion temperature and thus activate the temperature controlling device 118.

The base 102 con also be provided with a display on which the stored features, i.e. temperature, infusion time, etc., can be shown.

In the central unit can also be stored for a certain number of different beverages the desired value of particular parameters such as the optimal quantity of powder to be used, the optimal infusion temperature, the optimal infusion time, and the optimal quantity of water to pour in the container 2, as shown in the following table 1 by way of non-limitative example, for various tea blends.

TABLE 1

| | POWDER | WATER | WATER | INFUSION | |
|---|---|---|---|---|---|
| TEA BLEND | QUANTITY | TEMPERATURE | QUANTITY | TIME | |
| White tea - YIN ZEN | 5 g | 70° C. | 20 cl | 15 | min |
| White teas - PAI MU | 5 g | 85° C. | 20 cl | 7 | min |
| TAN | | | | | |
| Superior green teas | 5 g | 70° C. | 20 cl | 3 | min |
| Green teas | 5 g | 95° C. | 20 cl | 3 | min |
| Japanese green teas | 5 g | 90° C. | 20 cl | 1-2 | min |
| Blue teas | 5 g | 95° C. | 20 cl | 7 | min |
| Spring darjeeling | 5 g | 95° C. | 20 cl | 3 | min |
| Ripe black teas, with | 5 g | 95° C. | 20 cl | 5 | min |
| entire leaves FOP, OP, | | | | | |
| SOUCHONG | | | | | |
| BOP and BPS black | 5 g | 95° C. | 20 cl | 3 | min |
| teas, and PEKOE | | | | | |
| black teas | | | | | |
| BOPF black teas, with | 5 g | 95° C. | 20 cl | 2 | min |
| minced leaves | | | | | |
| Scented teas | 5 g | 95° C. | 20 cl | 3-5 | min |

Obviously similar table can be prepared also for different kind of beverages, for example for coffee beverages obtained with different blends, and/or with different features (strong coffee, weak coffee, etc.).

Optimal infusion time can be stored for obtaining weak coffee beverage and/or strong coffee beverage, for example a 2 min value for the infusion time for a weak coffee beverage and 4 min value for the infusion time for a strong coffee beverage.

The afore mentioned parameters can be stored in the central unit of the apparatus 100, for the various beverages, by the builders of the apparatus 100, or also directly by the user. It is also possible to store in the central unit the values of the afore mentioned parameters both for weak and medium and strong beverage, so that a user can always from time to time, for each kind of beverage he desires to prepare, to obtain exactly the beverage he desires.

The user can also store in the central unit the parameters for obtaining preferred beverages.

A selecting device can be provided in the base 102, for automatically select a particular beverage to be obtained amongst the stored beverages and to correspondingly program the controlling arrangement 111.

A quick selecting device can be provided in the base 102, for automatically and quickly select a particular preferred beverage to be obtained amongst the preferred stored beverages.

A user desiring to prepare a certain beverage, with particular features, has only to look for such beverage amongst the stored beverages and to choose the corresponding string of values so as to correspondingly program the apparatus 100.

Obviously, in the controlling arrangement 111, the temperature controlling device 118 and the timer 117 can be used simultaneously, so that a better control of the functioning of the apparatus 100 can be reached.

Using the apparatuses 1, 100 according to the invention beverages with high organoleptical feature can be obtained.

The apparatus 1, 100 can be further shaped in any desired manner decided on aesthetical or design's choices, in particular the container 2 can be made in non transparent material.

In the latter case, using an indicating device becomes particularly important because otherwise it would be impossible for a user to evaluate the features of the prepared beverage.

The tisane-pot 20 in FIGS. 3 and 4 comprises a cup 21 into which hot water is poured for preparing the beverage, a strainer 22 shaped so as an upper portion thereof 23 rests on an edge 24 of the tisane-pot, and so as that in use the strainer is located inside the cup 21, and arranged for receiving a desired quantity Q of herbal, phytotherapic preparations. The tisane-pot 20 comprises a lid 25 suitable for closing the cup 21 during the infusion of the phytotherapic preparations, and a handle 26 suitable for allowing the tisane-pot 20 to be handled even when the cup 21, and in particular the outer surface 27 thereof reaches high temperatures due to the presence of hot water into the cup 21.

To the tisane-pot 20 can be fixed in a stable or movable manner, a timer 17, in any suitable desired zone, for example on the handle 26, as indicated by the arrows F, or on any zone Z of the pouter surface of the cup 21.

1-136. (canceled)

137. Apparatus for obtaining beverages by infusion comprising at least one indicating device arranged for indicating that a preset infusion time has passed.

138. Apparatus according to claim 137, wherein said at least one indicating device comprises a regulating device) arranged for regulating the infusion time.

139. Apparatus according to claim 138, wherein said regulating device can be suitably regulated and programmed by a user.

140. Apparatus according to claim 138, wherein said regulating device comprises a switch arranged for alternately activating and/or deactivating said regulating device so as to automatically regulate the infusion time.

141. Apparatus according to claim 137, wherein said indicating device comprises a sensing device arranged for sensing a certain parameter and for automatically activating said indicating device.

142. Apparatus according to claim 141, wherein said sensing device comprises at least one temperature sensor sensing the temperature in said apparatus and activating said indicating device.

143. Apparatus according to claim 141, wherein said sensing device comprises at least one level sensor sensing the level of the liquid in said apparatus and activating said indicating device.

144. Apparatus according to claim 141, wherein said sensing device comprises at least one colorimetric sensor sensing the colour of the liquid in said apparatus and activating said indicating device.

145. Apparatus according to claim 141, wherein said sensing device comprises at least one transmittance sensor suitable for sensing the transmittance of the liquid in said apparatus and for activating said indicating device.

146. Apparatus according to claim 141, wherein said sensing device comprises at least one conductibility sensor for sensing the conductibility of the liquid in said apparatus and activating said indicating device.

147. Apparatus according to claim 141, wherein said sensing device comprises at least one sensor suitable for sensing the concentration of solutes present in the liquid in said apparatus and for activating said indicating device.

148. Apparatus according to claim 137, wherein said indicating device comprises at least one warning element arranged for warning a user that a preset value of a parameter has been reached in the liquid in a container of the apparatus.

149. Apparatus according to claim 141, wherein said sensing device comprises at least one further warning element suitable for warning a user that a preset value of a parameter has been reached in the liquid in a container of the apparatus.

150. Apparatus according to claim 137, wherein said indicating device comprises at least one warning element arranged for warning a user that a preset infusion time has been reached.

151. Apparatus according to claim 148, wherein said at least one warning element comprises a sound warning element emitting a sound signal for warning the user.

152. Apparatus according to claim 148, wherein said at least one warning element comprises a light warning element emitting a light signal for warning the user.

153. Apparatus according to claim 137, wherein said indicating device comprises a hooking element arranged for movably hooking said indicating device to said apparatus.

154. Apparatus according to claim 153, wherein said hooking element is selected in a group comprising: a Velcro hooking device, tongs, pliers, elastic band, magnets.

155. Apparatus according to claim 137, wherein said indicating device comprises a first device portion stably fixed to said apparatus, and a second device portion that can be movably associated to said first device portion.

156. Apparatus according to claim 137, wherein said apparatus is a coffee maker of the type known as “French Press”.

157. Apparatus according to claim 137, wherein said beverage is obtained starting from powdered substances, said powdered substances being selected from a group comprising: coffee, tea, camomile, flower-based blends, plant-based blends, phytotherapic mixtures, herbal preparations.

158. Apparatus according to claim 137, and further comprising a chamber arrangement for keeping almost constant the temperature into the apparatus.

159. Apparatus according to claim 137 and further comprising a frothing device arranged for frothing the content in a container of the apparatus.

160. Apparatus according to claim 159, wherein said frothing device comprises a magnetic stirring device.

161. Apparatus according to claim 137 and further comprising a heating device arranged for heating the content in a container of the apparatus.

162. Apparatus according to claim 137, and further comprising a connecting element for connecting said apparatus to a base arranged for receiving said apparatus.

163. Apparatus according to claim 162, wherein said base comprises a heating device arranged for heating the content in a container of the apparatus.

164. Apparatus according to claim 162, wherein said base comprises a controlling arrangement arranged for controlling the functioning of the apparatus.

165. Apparatus according to claim 164, wherein said controlling arrangement comprises at least one indicating device arranged for indicating a value of a desired functioning parameter of said apparatus.

166. Apparatus according to claim 164, wherein said controlling arrangement further comprises a temperature controlling device for controlling the temperature in a container of the apparatus.

167. Apparatus according to claim 164, wherein said controlling arrangement further comprises a temperature indicating device for indicating the temperature in a container of the apparatus.

168. Apparatus according to claim 164, wherein said controlling arrangement further comprises a time indicating device for indicating the infusion time in a container of the apparatus.

169. Apparatus according to claim 164, wherein said controlling arrangement further comprises a frothing controlling device for controlling a frothing device for frothing the content in a container for producing foam.

170. Method for obtaining beverages by infusion comprising imbibing a dose of a substance or of a mixture of substances from which said beverages have to be obtained with hot water at a desired temperature, and indicating the duration of the infusion by means of suitable indicating device indicating that a preset infusion time has passed.

171. Timing device provided with a hooking element arranged for hooking said timing device to an apparatus for preparing beverages by infusion.

172. A base comprising a connecting element for connecting said base to a container for preparing beverages by infusion and a heating device for heating a content of said container.
